(19) United States
(12) Patent Application Publication
(10) Pub. No.: US 2019/0022618 A1
(43) Pub. Date: Jan. 24, 2019

(54) SURFACE TREATED LIPID SUPPORTED MULTILAYERS

(71) Applicant: The Florida State University Research Foundation,, Tallahassee, FL (US)

(72) Inventors: Steven J. Lenhert, Tallahassee, FL (US); Aubrey E. Kusi-Appiah, Tallahassee, FL (US)

(21) Appl. No.: 16/038,450

(22) Filed: Jul. 18, 2018

Related U.S. Application Data
(60) Provisional application No. 62/534,012, filed on Jul. 18, 2017.

(51) Int. Cl. B01J 19/00; C12N 5/00

(52) U.S. Cl. CPC B01J 19/0046; C12N 5/00; B01J 2219/00605; G03F 7/0002; B01J 2219/00677; C12N 2539/00; B01J 2219/00734

(57) ABSTRACT

Disclosed herein are devices comprising treated lipid multilayer arrays. The devices can include a support, a discrete lipid multilayer array on a surface of the support, wherein the lipid multilayer array comprises one or more lipid multilayer dots, a material encapsulated in the one or more lipid multilayer dots, and a silicon containing compound present on a surface of one or more of the lipid multilayer dots. In some embodiments, the encapsulated material is a hydrophilic small molecule. The devices disclosed herein exhibit increased stability in cell-based applications, such as under high protein cell culture media, as well as allow for viable cell adhesion. Methods for making the disclosed devices are also provided.

### CROSS-REFERENCE TO RELATED APPLICATIONS

This application claims the benefit of and priority to U.S. Patent Application No. 62/534,012 filed on Jul. 18, 2017, the disclosure of which is expressly incorporated herein by reference in its entirety.

### ACKNOWLEDGEMENT OF GOVERNMENT SUPPORT

This invention was made with government support under Grant No. NIH R01 GM107172 awarded by the National Institutes of Health. The government has certain rights in the invention.

### FIELD OF THE DISCLOSURE

This disclosure relates generally to lipid multilayer arrays, particularly to surface treated lipid multilayer arrays.

### BACKGROUND OF THE DISCLOSURE

Fluid phospholipids have demonstrated their usefulness in biotechnology and biomimetic applications such as cell modeling and drug delivery (Bailey, et al., Proc. Natl. Acad. Sci. U. S. A. 101, 16144-16149 (2004); Kusi-Appiah, et al., Lab on a Chip 15, 3397-3404 (2015); and Kusi-Appiah, et al., Biomaterials 33, 4187-4194 (2012)). However, their exploitation has been limited to low throughput applications due to the instability of supported lipid multilayers (SLM) to aqueous immersion. This instability can occur as a result of dissolution of the phospholipids at the air-water-lipid interface, causing the lipids to be carried along with the solution (FIG. 1B shows the fluorescence image of the destruction of lipid multilayer patterns when immersed in cell culture media). Attempts have been made to stabilize SLM patterns by reducing the humidity of the immersion environment and using hydrophobic surfaces like poly(methyl methacrylate) (Lenhert, et al., Nature Nanotechnology 5, 275-279 (2010)). While these attempts have been successful for immersion of the SLMs in simple buffers, immersion under high-protein-content media has remained a challenge.

There is a need for lipid multilayer arrays that are stable in cell-based applications. There is also a need for lipid multilayer arrays that are stable under high protein cell culture media. There is still a need for lipid multilayer arrays that allow for viable cell adhesion. The devices and methods disclosed herein address these and other needs.

### SUMMARY OF THE DISCLOSURE

Disclosed herein are devices comprising one or more patterned arrays of treated lipid multilayer dots. The devices can include a support, a discrete lipid multilayer array on a surface of the support, wherein the lipid multilayer array comprises one or more lipid multilayer dots, a material encapsulated in the one or more lipid multilayer dots, and a silicon containing compound present on a surface of the one or more lipid multilayer dots. The lipid multilayer dot can have any suitable size. In some embodiments, the lipid multilayer dot has a height of 50 μm or less, such as from 10 nm to 50 μm or from 10 nm to 10 μm. In some embodiments, the devices disclosed herein, after submerged in water for 100 minutes at from 25° C. to 37° C., exhibit a leakage of less than 15 wt % of the material originally encapsulated in the lipid multilayer structure.

The silicon containing compound present on the surface of the lipid multilayer dot can be selected from a silica based compound. The silica based compound can be derived from an alkyl silicate such as tetramethyl orthosilicate, tetraethyl orthosilicate, tetraisopropyl orthosilicate, tetrapropyl orthosilicate, or combinations thereof. In some embodiments, the silicon containing compound forms a lipid-silicon based hybrid assembly on the surface of the lipid multilayer dot. The silicon containing compound can be present in an amount of from greater than 0 wt % to 70 wt %, such as from 5 wt % to 50 wt %, based on the weight of the lipid multilayer dot.

As disclosed herein, the lipid multilayer dot can include an encapsulated material. The encapsulated material can be a hydrophilic or a hydrophobic material. In some embodiments, the encapsulated material can be selected from a hydrophilic material, such as a hydrophilic drug.

The lipid multilayer can further include a labeling material or a targeting agent, which may be present on a surface of the lipid multilayer dot.

In some embodiments, the devices disclosed herein can include a plurality of lipid multilayer arrays. In specific examples, the device can include a second lipid multilayer array, wherein the second lipid multilayer array comprises one or more second lipid multilayer structure, and wherein the one or more second lipid multilayer structure encapsulates a second material.

Methods of making the disclosed devices are also provided. The method can include depositing a lipid multilayer array on a surface of a support, wherein the lipid multilayer array comprises one or more lipid multilayer dots, contacting a surface of the lipid multilayer a with a silicon containing precursor, and reacting the silicon containing precursor to form a silica based coating on the lipid multilayer dot. The silicon containing precursor can be in the form of a solution or vapor. In some embodiments, the step of contacting the surface of the lipid multilayer dot with the silicon containing precursor can be performed at room temperature.

In some examples, the method of producing the device includes depositing one or more lipid droplets on a surface at a temperature of from −10° C. to 30° C., wherein the lipid droplet comprises a therapeutic material and a silicon containing coating; storing the one or more lipid droplets at a temperature of 10° C. or less for a period of at least 10 minutes (such as from 10 minutes to 48 hours); printing using a nanointaglio process the one or more lipid droplets on a substrate using a topographically structured stamp within five minutes of exposure to a temperature above 10° C.; and removing the stamp from the substrate to form a patterned substrate. In some embodiments, the stamp can be derived from polydimethylsiloxane (PDMS).

Method for delivering an encapsulated material such as a hydrophilic material using the devices disclosed herein are also provided. In some embodiments, the methods can include providing a lipid multilayer array comprising a lipid multilayer dot as disclosed herein and delivering the encapsulated material to a cell from the lipid multilayer dot that is in contact with the cell.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIGS. 1A-1F show fluid lipid multilayers are unstable to aqueous immersion and cell culture media environments but can be stabilized by treatment with TEOS. FIG. 1A is an in-air fluorescence image of lipid multilayer arrays doped with rhodamine-PE; FIG. 1B is a fluorescence image of rhodamine-PE doped fluid lipid multilayers showing destruction upon immersion in cell culture media even under low humidity conditions. FIG. 1C is a graph showing quantification of multilayer destruction upon immersion under cell culture media. The (*) indicates significant difference in measured destruction from control multilayers in air (p<0.05). FIG. 1D is an in-air fluorescence image of untreated lipid multilayer arrays doped with rhodamine-PE. Inset is of selected area in the smaller white box with arrows showing a few areas of non-uniformity. FIG. 1E is a fluorescence image of TEOS-treated lipid multilayer arrays doped with rhodamine-PE and immersed under water. Inset shows selected area in the white box. The arrows in FIG. 1B and FIG. 1D indicate direction of flow of water during the immersion process. FIG. 1F is a graph showing the measured uniformity of the lipid multilayers before TEOS treatment and under water post immersion. Lipid used here is DOPC.

FIGS. 2A-2C show TEOS-treated lipid multilayers are stable to AFM imaging. FIGS. 2A and 2B are AFM peak force mode of the same TEOS-treated fluid lipid multilayers pre- and post-immersion under HBSS buffer. FIG. 2C is a graph of the AFM-measured heights of TEOS-treated lipid multilayers pre-immersion versus immediately post-immersion. Slope=1.1877, R²=0.9939.

FIGS. 3A-3D show TEOS-treated lipid multilayers crack with extended exposure to water. FIG. 3A shows TEOS-treated lipid multilayers show visible cracks after ˜2 hours of immersion under water. FIG. 3B is a sample AFM micrograph of TEOS-treated lipid multilayers showing visible cracks in the dots after exposure to ˜60% humidity for >2 hours. FIG. 3C is a schematic of lipid multilayers with silica-phospholipid hybrid outer shell in air. FIG. 3D is a schematic of humidity-induced cracking of the silica-phospholipid hybrid outer shell of the lipid multilayers in high humidity or liquid water.

FIGS. 4A-4D show different cell types adhere to TEOS-treated lipid multilayers. FIG. 4A shows a merged fluorescence and bright field images showing Hela cells growing over DOPC multilayers doped with rhodamine-PE. FIG. 4B shows a merged fluorescence and bright field images showing Hela cells growing over TEOS treated DOPC multilayers doped with rhodamine-PE. FIG. 4C shows a merged fluorescence image showing cell nuclei (˜15 μm) adhered over the rhodamine-PE doped TEOS-treated DOPC multilayers (5 μm). FIG. 4D is a graph showing different cells adhere to different degrees onto the TEOS-treated lipid multilayers. The (*) indicates a significant difference from the area without lipid for that cell type (p<0.05). Experiments were performed in triplicate.

FIGS. 5A-5B are schematic diagrams showing encapsulation of hydrophilic molecules in oil (FIG. 5A) and liposomes (FIG. 5B) for microarray printing.

FIG. 6 shows TEOS/TMOS stabilization of lipid multilayers with encapsulated hydrophilic drugs.

FIG. 7 is a schematic diagram showing encapsulation of hydrophobic molecules for microarray printing.

FIG. 8 shows TEOS/TMOS stabilization of lipid multilayers.

FIGS. 9A-9C show in-air fluorescence image of untreated lipid multilayer arrays doped with rhodamine-PE. Inset is of selected area in the smaller white box (FIG. 9A); fluorescence image of TEOS-treated lipid multilayer arrays doped with rhodamine-PE and immersed under water. Inset shows selected area in the white box—the arrow indicates direction of flow of water during the immersion process (FIG. 9B); and a graph showing the measured uniformity of the lipid multilayers before TEOS treatment and under water post immersion (FIG. 19C).

FIGS. 10A-10C show TEOS-treated lipid multilayers are stable to AFM imaging. FIGS. 10A and 10B are AFM peak force mode of the same TEOS-treated fluid lipid multilayers pre- and post-immersion under HBSS buffer. FIG. 10C is a graph of the AFM-measured heights of TEOS-treated lipid multilayers pre-immersion versus immediately post-immersion. Slope=1.1877, R²=0.9939.

FIG. 11 shows TEOS/TMOS treatment prevents leakage of both hydrophobic and hydrophilic encapsulated molecules. Both hydrophilic and hydrophobic rhadamine dyes remain encapsulated in the lipid multilayers over an hour and half while encapsulated hydrophilic rhodamine leaks from the untreated lipid multilayers. TMOS treatment also mitigates leakage of hydrophilic molecules like free rhodamine dye from oil multilayers such as the mixture of castor oil and hexanoic acid (Hex-Cas) used here.

FIGS. 12A-12C show iSLK.219 assay is compatible with lipid microarray screening, including surface delivery of water soluble doxycycline. FIG. 12A is a fluorescence micrograph of an array prior to cell culture. Only the right column of spots contains doxocyclin. FIG. 12B is a fluorescence micrograph of the array after cell culture. FIG. 12C is a higher magnification of the area highlighted by the rectangle in FIG. 12B showing DAPI stained nuclei and the two reporter genes.

### DETAILED DESCRIPTION

Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise. The disclosure of percentage ranges and other ranges herein includes the disclosure of the endpoints of the range and any integers provided in the range.

As used herein, the term “array” refers to a one-dimensional or two-dimensional set of microstructures, such as dots. An array may have any shape. For example, an array may be a series of microstructures arranged in a line, such as an array of squares. An array may be arranged in a square or rectangular grid. There may be sections of the array that are separated from other sections of the array by spaces. An array may have other shapes. For example, an array may be a series of microstructures arranged in a series of concentric circles, in a series of concentric squares, in a series of concentric triangles, in a series of curves, etc. The spacing between sections of an array or between microstructures in any array may be regular or may be different between particular sections or between particular pairs of microstructures. The microstructure arrays of the present invention may be comprised of microstructures having zero-dimensional, one-dimensional or two-dimensional shapes. The microstructures having two-dimensional shapes may have shapes such as squares, rectangles, circles, parallelograms, pentagons, hexagons, irregular shapes, etc.

As used herein, the term “dot,” also referred to herein as lipid multilayer dot, refers to an individual lipid multilayer microstructure of an array. The lipid multilayer dots are surface supported (that is, disposed on a surface of a support), a plurality of which are separated by spaces on the support. The devices disclosed herein can include a patterned array of 2 or more, 5 or more, 10 or more, 50 or more, 100 or more, 1,000 or more, 5,000 or more, of 10,000 or more dots.

As used herein, the term “biomolecule” refers to the conventional meaning of the term “biomolecule,” i.e., a molecule produced by or found in living cells, e.g., a protein, a carbohydrate, a lipid, a phospholipid, a nucleic acid, etc.

As used herein, the term “drug” refers to any chemical substance that affects the functioning of a cell. A drug may be natural or synthetic. Although only particular drugs are described as being used in the examples below, almost any type of drug may be used in the embodiments of the present invention. For example, a drug may be a biomolecule. A drug may be tagged with a marker, such as a fluorescent marker, a radioactive marker, etc. to allow the drug to be tracked in an assay.

As used herein, the term “deliver” refers to the transfer of an encapsulated material, such as a drug, from a lipid multilayer structure to a cell in contact with the structure. An encapsulated material may be “delivered” by various means. In one embodiment of the present invention, an encapsulated material is delivered to a cell by the cell taking up a dot (lipid multilayer microstructure) that encapsulates the encapsulated material. The dot is part of an array on a substrate and the cell takes up the dot by direct contact with the dot and fusion of the dot with the cell membrane by endocytosis.

As used herein, the term “encapsulate” refers to the process of loading a material, such as a drug, that is contained in, confined by or otherwise held by a lipid multilayer structure. A portion of an encapsulated material may protrude from a lipid multilayer structure and still be encapsulated by structure.

As used herein, the term “encapsulated material” refers to any material that is encapsulated in a lipid multilayer structure. Examples of encapsulated materials include drugs; small molecules, such as drug candidates; lipid additives, such as functionalized phospholipids or cholesterol; larger molecules, such as nucleic acids including DNA, RNA, etc., different from peptides, proteins, etc.; microparticles, nanoparticles. An encapsulated material may be tagged with a marker, such as a fluorescent marker, a radioactive marker, etc. to allow the encapsulated material to be tracked in an assay.

As used herein, the term “therapeutic agent” or “drug” refers to any moiety that is a biologically, physiologically, or pharmacologically active substance that acts locally or systemically in a subject. The therapeutic agent can be a small molecule or a macromolecule. Some examples of therapeutic agents are described in well-known literature references such as the Merck Index, the Merck manual of diagnosis and therapy, the Physicians Desk Reference, and The Pharmacological Basis of Therapeutics. They include in the list metal binding proteins, peptides, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. The “therapeutic agent” or “drug” described herein include “potential therapeutic agent” or “drug candidates,” which include small organic molecules (typically with a molecular weight below 1000 Da), antibodies, antibody fragments and therapeutic proteins and peptides. The small molecules may belong to any chemical class suspected to interact with a macromolecule such as a protein and expected to be pharmaceutically acceptable. Antibodies may belong to any of the immunoglobulin (Ig) classes, e.g. IgA, IgD, IgE, IgG or IgM, and may be polyclonal, monoclonal, genetically engineered, e.g. humanized, or otherwise adapted to a particular use. Antibody fragment may be e.g. a heavy chain, light chain, Fab or Fc fragment, or single chain fragment, such as scFv. Therapeutic proteins or peptides may be any protein or peptide in its natural, modified natural or fully recombinant form.

As used herein, the term “lipid” refers to the conventional meaning of the term “lipid.” Lipids include fats, waxes, sterols, oils, fat-soluble vitamins (such as vitamins A, D, E, and K), monoglycerides, diglycerides, triglycerides, phospholipids, etc.

As used herein, the term “lipid multilayer” refers to a lipid coating that is thicker than a single bilayer (>5 nm).

As used herein, the term “lipid multilayer array” refers to an array comprising lipid multilayer structures.

As used herein, the term “lipid multilayer structure” refers to a structure comprising one or more lipid multilayers.

As used herein, the term “microstructure” refers to a structure having at least one dimension smaller than 1 mm. A nanostructure is one type of microstructure.

As used herein, the term “nanostructure” refers to a structure having at least one dimension on the nanoscale, i.e., a dimension between 0.1 and 100 nm.

As used herein, the term “plurality” refers to two or more. Therefore, an array of microstructures having a “plurality of heights” is an array of microstructures having two or more heights. However, some of the fluorescent microstructures in an array having a plurality of heights may have the same height.

As used herein, the term “nanointaglio” refers to a printing process by which ink is transferred from recesses of a stamp resulting in topographically structured ink patterns. See for example, Lowry et al., Advanced Materials Interfaces, 2014, 1 (4). The nanoscale control of lipid ink droplet topography and volume afforded by an intaglio stamp, in combination with pin-spotting, enables lipid arrays to demonstrate size-dependent functionality. The scalable, multi-integrative capabilities of nanointaglio have potential applications in high throughput screening and biosensor arrays.

Devices

Screening the effects of small molecules on cells grown in culture is a well-established method for drug discovery and testing, and faster throughput at lower cost is needed. Small-molecule arrays and microfluidics are promising approaches. Disclosed herein are devices comprising a microarray of lipid multilayers. The devices can provide a surface-mediated delivery of drugs to cells from the microarray of lipid multilayers encapsulating drug. The multilayer patterns can be of sub-cellular dimensions and controllable thickness and can be formed by dip-pen nanolithography. The patterns can successfully delivered a small molecule only to the cells directly over them, indicating successful encapsulation and no cross-contamination to cells grown next to the patterns.

In some embodiments, the devices disclosed herein comprises treated lipid multilayers. For example, the devices can include a support, a discrete lipid multilayer dot on a surface of the support, an encapsulated material, and a silicon containing compound present on a surface of the lipid multilayer dot.

Silicon Containing Compound

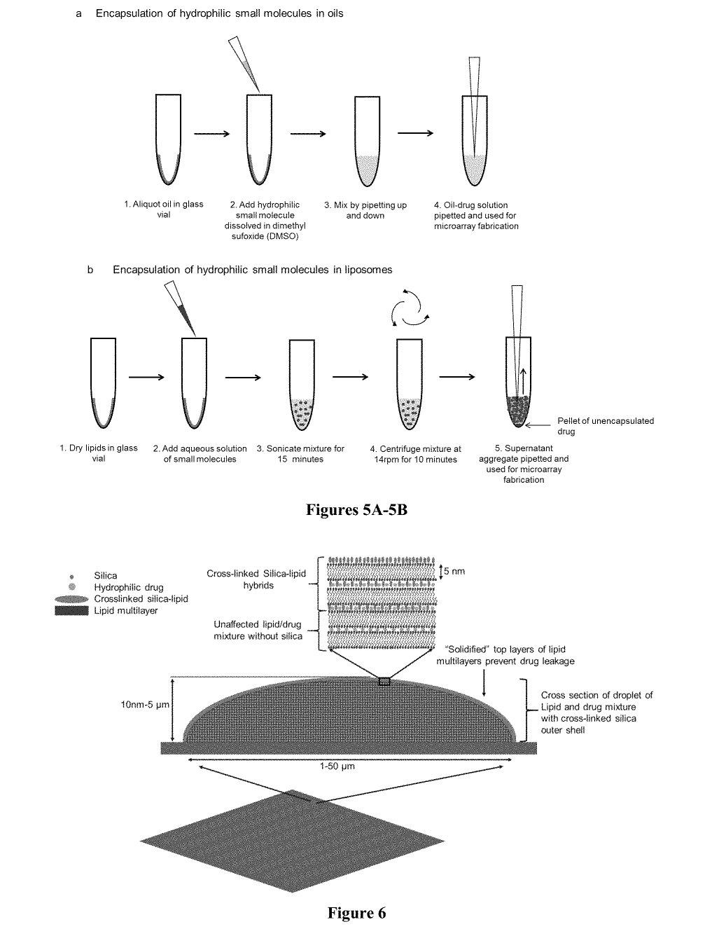

As described herein, the lipid multilayer dot can comprise a silicon containing compound on a surface thereof. The silicon containing compound may form a hybrid lipid-silicon based assembly on the surface of the lipid multilayer dot. The pattern of the hybrid assembly can be described as a lamella phase structure with alternating lipid and silicon containing compound as shown in FIGS. 6 and 8. The lipid headgroup can associate with the silicon containing compound through hydrogen bonding or dipole interactions. The charge and/or polarity of the lipid headgroup may affect hybrid formation.

The silicon containing compound present on the lipid multilayer dot can be silica based. The silica based compound can be derived from a precursor, for example, a silicate precursor. Examples of silicate precursors include alkyl silicates, such as tetramethyl orthosilicate, tetraethyl orthosilicate, tetraisopropyl orthosilicate, tetrapropyl orthosilicate, trialkoxysilanes such as aminopropyltrimethyoxysilane, hydroxymethyltriethoxysilane, methacryloxypropyl trimethoxysilane, or combinations thereof. The silicon containing compound on the surface of the lipid multilayer dot may also be derived from silicic acid; inorganic silicates such as an alkali or ammonium silicate; inorganic fluorosilicates; silicon tetrahalide; or organic orthosilicates such as tetraalkylammoniumsilicates.

The silicon containing compound can be present in an amount of from greater than 0% to 70% by weight of the lipid multilayer dot (i.e. including the lipid multilayer structure, the silicon containing compound, and the encapsulated material). For example, the silicon containing compound can be present in an amount of 0.5% or greater, 1% or greater, 1.5% or greater, 2% or greater, 5% or greater, 8% or greater, 10% or greater, 15% or greater, 20% or greater, 25% or greater, 30% or greater, 35% or greater, 40% or greater, 45% or greater, 50% or greater, 55% or greater, 60% or greater, or 65% or greater by weight, based on the total weight of the lipid multilayer dot. In some examples, the silicon containing compound can be present in an amount of 70% or less, 65% or less, 60% or less, 55% or less, 50% or less, 45% or less, 40% or less, 30% or less, 25% or less, 20% or less, 15% or less, 40% or less, 5% or less, or 2% or less by weight, based on the total weight of the lipid multilayer dot. The amount of silicon containing compound in the devices described herein can range from any of the minimum values described above to any of the maximum values described above. For example, the amount of silicon containing compound in the device can range from 0.1% to 70%, 1% to 70%, 1% to 50%, 5% to 50%, 1% to 25%, or 1% to 30% by weight, based on the total weight of the lipid multilayer dot.

Lipids

The lipid multilayer dot can be derived from any suitable lipid. The lipid can be selected from saturated or unsaturated lipids, a small molecular lipid, a macromolecular lipid, or a polymeric lipid. The selection of a particular lipid and the concentration of the lipid may depend in part on the type of resulting multilayer and micro- or nano-structures to be obtained. In some cases, the lipid may be water-insoluble, biocompatible and enzymatically biodegradable in vivo to enable gradual exposure of the matrix and hence release of components within.

Suitable lipids for use in the lipid multilayer dots can include fatty acids such as hexanoic acid, stearic acid, 12-hydroxystearic acid, and oleic acid; vegetable oils; beeswax; glycerol behenate; castor oil; soybean oils; phospholipids; lecithin; and mixtures thereof. Specific examples of phospholipids for use within the lipid multilayer dots can include 1,2-dimyristoyl-sn-glycero-3-phosphocholine (DMPC; 14:0); 1,2-dimyristoyl-sn-glycero-3-phosphoethanolamine (DMPE; 14:0); 1,2-dimyristoyl-sn-g/ycero-3-[phospho-rac-(1-glycerol)] (Sodium Salt)(DMPG, 14:0); 1-myristoyl-2-hydroxy-sn-glycero-3-phosphocholine (14:0 Lyso PC); 1,2-dioleoyl-sn-glycero-3-phosphocholine (DOPC; 18:1 (cis)); 1-oleoyl-2-hydroxy-sn-glycero-3-phosphoethanolamine (18:1 Lyso PE); 1,2-dioleoyl-sn-glycero-3-phosphoethanolamine (DOPE, 18:1); L-phosphatidylcholine (egg, soy); phosphatidylcholine (NBD); 1,1′,2,2′-tetramyristoyl cardiolipin (ammonium salt) (14:0); lipids with head groups phosphatidyl serine and phosphatidylinositol; poly(ethylene glycol)-lipid conjugates; and fluoroscent lipids-phosphatidylcholine (NBD) or combinations thereof.

The lipid can be present in an amount of from greater than 0% by weight to 50% by weight of the lipid multilayer dot (including the lipid multilayer structure, the silicon containing compound, and the encapsulated material). For example, the lipid can be present in an amount of 0.5% or greater, 1% or greater, 1.5% or greater, 2% or greater, 5% or greater, 8% or greater, 10% or greater, 15% or greater, 20% or greater, 25% or greater, 30% or greater, 35% or greater, 40% or greater, 45% or greater, or 50% or greater by weight, based on the total weight of the lipid multilayer dot. In some examples, the lipid can be present in an amount of 50% or less, 45% or less, 40% or less, 30% or less, 25% or less, 20% or less, 15% or less, 40% or less, 5% or less, or 2% or less by weight, based on the total weight of the lipid multilayer dot. The amount of lipid in the devices described herein can range from any of the minimum values described above to any of the maximum values described above. For example, the amount of lipid in the devices can range from 0.1% to 50%, 1% to 50%, 5% to 45%, 5% to 40%, 10% to 50%, or 10% to 45% by weight, based on the total weight of the lipid multilayer dot. The concentration of the lipids may affect the spacing of the structures present in the lipid multilayer dot.

Encapsulated Material

The lipid multilayer dots can encapsulate both hydrophilic and lipophilic materials. In some embodiments, the encapsulated material can be a therapeutic agent, in particular, active ingredients acting in a chemical, biological or physical manner. In specific embodiments, the lipid multilayer dot can encapsulate a hydrophilic material such as a hydrophilic therapeutic agent. The term hydrophilic, as used herein, refers to a material having an octanol-water partition coefficient (Log P) of about 1.5 or less, 1.0 or less, or 0.5 or less. The octanol-water partition coefficient (Log P) is calculated in accordance with the following method. First, a molecule is dissolved in a mixed solution (1:1) of octanol and water. When phase separation takes place, concentrations of the drug dissolved in each phase are measured. Logarithms are taken on the relative value of the measured concentrations to calculate a partition coefficient (Log P) of the drug, which is given by the equation below:

Log P=Log (C_{octanol}/C_{water}) wherein C_{octanol} represents the concentration of the drug dissolved in the octanol layer, and C_{water} represents the concentration of the drug dissolved in the water layer. The lower the Log P value is, the higher the hydrophilicity of a molecule. In some cases, the hydrophilic material can have a water solubility at 25° C. of greater than 1 g/1 kg of water.

The term “therapeutic agent” encompasses drugs, genetic materials, and biological materials. For example, the therapeutic agent may be useful for inhibiting cell proliferation, contraction, migration, hyperactivity, or addressing other conditions. Examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti thrombin antibodies, anti platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. In a preferred embodiment, the therapeutic agent is taxol (e.g., Taxol®), or its analogs or derivatives. In another preferred embodiment, the therapeutic agent is paclitaxel. In yet another preferred embodiment, the therapeutic agent is an antibiotic such as erythromycin, amphotericin, rapamycin, adriamycin, and such the like.

The term “genetic materials” refer to DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include anti-thrombogenic agents such as heparin, heparin derivatives, urokinase, and PPack (dextrophenylalanine proline arginine chloromethylketone); viral activation molecules like doxycycline which activates viral expression in Karposi Sarcoma endothelial (iSLK) cells; anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin; anti-inflammatory agents such as glucocorticoids, betamethasone, dexamethasone, prednisolone, corticosterone, budesonide, estrogen, sulfasalazine, rosiglitazone, mycophenolic acid and mesalamine; anti-neoplastic/anti-proliferative/anti-miotic agents such as paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, taxol and its analogs or derivatives; anesthetic agents such as lidocaine, bupivacaine, and ropivacaine; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides; DNA demethylating drugs such as 5-azacytidine, which is also categorized as a RNA or DNA metabolite that inhibit cell growth and induce apoptosis in certain cancer cells; vascular cell growth promoters such as growth factors, vascular endothelial growth factors (VEGF, all types including VEGF-2), growth factor receptors, transcriptional activators, and translational promoters; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vasoactive mechanisms; anti-oxidants, such as probucol; antibiotic agents, such as penicillin, cefoxitin, oxacillin, tobranycin, macrolides such as rapamycin (sirolimus) and everolimuns; angiogenic substances, such as acidic and basic fibroblast growth factors, estrogen including estradiol (E2), estriol (E3) and 17-beta estradiol; and drugs for heart failure, such as digoxin, beta-blockers, angiotensin-converting enzyme (ACE) inhibitors including captopril and enalopril, statins and related compounds. Preferred biologically active materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogues, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt. Other therapeutic agents include nitroglycerin, nitrous oxides, nitric oxides, antibiotics, aspirins, digitalis, estrogen derivatives such as estradiol and glycosides.

In some embodiments, the hydrophilic material can be a hydrophilic small molecule. In some embodiments, the hydrophilic material can be a hydrophobic small molecule. The term “small molecule,” as used herein, refers to a low molecular weight chemical compound, either natural or synthetic. Such small molecules may be a therapeutically deliverable substance or may be further derivatized to facilitate delivery. By “low molecular weight” is meant compounds having a molecular weight of less than 1000 Daltons, in some instances between 100 and 700 Daltons. Low molecular weight compounds, in various aspects, are about 100 or greater, about 150 or greater, about 200 or greater, about 250 or greater, about 300 or greater, about 350 or greater, about 400 or greater, about 450 or greater, about 500 or greater, about 550 or greater, about 600 or greater, about 650 or greater, about 700 or greater, about 750 or greater, about 800 or greater, about 850 or greater, about 900 or greater, or about 1000 or greater Daltons.

In some cases, the encapsulated material can include a labeling agent. The term “labeling agent” as used herein refers to a material which binds to the lipid multilayer array or a component thereof and is detectable by a physical or chemical method to permit identification of the location or quantity of the lipid multilayer array or a component thereof. Detection of the labeling material can be performed by any appropriate method known in the art. In some embodiments, the labeling agent can be a phosphor, which generally refers to a substance that is excited when irradiated with an X-ray, ultraviolet radiation, visible light, near-infrared radiation or the like from outside and emits light during the transition from the excited state back to the ground state. Accordingly, regardless of the mode of transition from the excited state back to the ground state, the “phosphor” in the present invention may be a substance that emits fluorescence in a narrow sense, which is light emission associated with deactivation from an excited singlet state, or may be a substance that emits phosphorescence, which is light emission associated with deactivation from a triplet state.

Examples of an labeling agents include substances known as organic fluorescent dyes, such as fluorescein-based dye molecules, rhodamine-based dye molecules, Alexa Fluor (registered trademark, manufactured by Invitrogen)-based dye molecules, BODIPY (registered trademark, manufactured by Invitrogen)-based dye molecules, Cascade (registered trademark, manufactured by Invitrogen)-based dye molecules, coumarin-based dye molecules, NBD (registered trademark)-based dye molecules, pyrene-based dye molecules, Texas Red (registered trademark)-based dye molecules, cyanine-based dye molecules, perylene-based dye molecules and oxazine-based dye molecules.

The encapsulated material can be present in an amount of from greater than 0% by weight to 50% by weight of the lipid multilayer dot (including the lipid multilayer structure, the silicon containing compound, and the encapsulated material). For example, the encapsulated material can be present in an amount of 0.5% or greater, 1% or greater, 1.5% or greater, 2% or greater, 5% or greater, 8% or greater, 10% or greater, 15% or greater, 20% or greater, 25% or greater, 30% or greater, 35% or greater, 40% or greater, 45% or greater, or 50% or greater by weight, based on the total weight of the lipid multilayer dot. In some examples, the encapsulated material can be present in an amount of 50% or less, 45% or less, 40% or less, 30% or less, 25% or less, 20% or less, 15% or less, 40% or less, 5% or less, or 2% or less by weight, based on the total weight of the lipid multilayer dot. The amount of encapsulated material in the devices described herein can range from any of the minimum values described above to any of the maximum values described above. For example, the amount of encapsulated material in the device can range from 0.1% to 50%, 1% to 50%%, 5% to 45%, 5% to 40%, 10% to 50%, or 10% to 45% by weight, based on the total weight of the lipid multilayer dot.

In some embodiments, the lipid multilayer dots can further include a labeling material or a targeting agent which may be encapsulated or present on a surface of the lipid multilayer dot. The term “targeting agent” as used herein means any moiety whose attachment to a substance allows the increase in concentration of the lipid multilayer array or a component thereof at a site of treatment, for example, a tumor site. Exemplary targeting agents include but are not limited to carbohydrates, peptides, vitamins, and antibodies.

Lipid Multilayer Dots and Arrays

The lipids, silicon containing compounds, and encapsulated material are used to form lipid multilayer dots and arrays. Lipid multilayer dots and multilayer arrays and methods of making and using are disclosed in U.S. Pat. Nos. 9,447,446 and 9,513,222, which are incorporated herein by reference in their entirety. The multilayer patterns can be of sub-cellular dimensions and controllable thickness and are formed by dip-pen nanolithography. In some embodiments, the present disclosure provides a combination of scalable pin-spotting microarray technology with the process of lipid multilayer stamping in order to generate nanostructured lipid multilayer microarrays suitable for cell culture applications such as screening of liposomal drug formulations on a chip. In some embodiments, the present disclosure provides a small-molecule microarray based on the use of lipid multilayer structures formed on surfaces by DPN. Molecules can be encapsulated within multilayer patterns of for example, phospholipids for delivery to cells.

The lipid multilayer array may have any shape. For example, an array may be a series of dot structures arranged in a line, such as the array of squares. An array may be arranged in a square or rectangular grid, such as the array of dots, such as shown in U.S. Patent Application No. 2012/0098974, which is hereby incorporated herein by reference. There may be sections of the array that are separated from other sections of the array by spaces, in which there are “sections,” for example, a rectangular grid arrays of dots, that are separated from each other by regular spacing. An array may have other shapes. For example, an array may be a series of dot structures arranged in a series of concentric circles, in a series of concentric squares, in a series of concentric triangles, in a series of curves, etc. The spacing between sections of an array or between dot structures in any array may be regular or may be different between particular sections or between particular pairs of dot structures. The arrays disclosed herein may be comprised of structures having zero-dimensional, one-dimensional or two-dimensional shapes. The dot structures having two-dimensional shapes may have shapes such as squares, rectangles, circles, parallelograms, pentagons, hexagons, irregular shapes, etc.

The height of the lipid multilayer dot may vary depending upon factors such as the degradation rate and the length of time the multilayer is required. For example, the lipid multilayer dot may vary in height from about 10 nm to 50 microns. In some embodiments, the multilayer can have a height 50 microns or less, 45 microns or less, 40 microns or less, 35 microns or less, 30 microns or less, 25 microns or less, 20 microns or less, 15 microns or less, 10 microns or less, 5 microns or less, 4 microns or less, 3 microns or less, 2 microns or less, 1 micron or less, 900 nm or less, 800 nm or less, 700 nm or less, 600 nm or less, 500 nm or less, 300 nm or less, 200 nm or less, 100 nm or less, or 50 nm or less. In some embodiments, the multilayer dot can have a height 10 nm or greater, 20 nm or greater, 30 nm or greater, 40 nm or greater, 50 nm or greater, 75 nm or greater, 100 nm or greater, 200 nm or greater, 500 nm or greater, 750 nm or greater, 1 micron or greater, 2 microns or greater, 3 microns or greater, 4 microns or greater, 5 microns or greater, 7 microns or greater, 8 microns or greater, 10 microns or greater, 15 microns or greater, 20 microns or greater, 30 microns or greater, or 40 microns or greater. In some embodiments, the multilayer dot can have a height of from 10 nm to 50 microns, from 10 nm to 40 microns, from 10 nm to 20 microns, from 10 nm to 10 microns, from 10 nm to 5 microns, from 10 nm to 2 microns, from 10 nm to 1 micron, from 50 nm to 50 microns, from 50 nm to 30 microns, from 50 nm to 10 microns, from 50 nm to 5 microns, or from 50 nm to 1 micron.

The diameter of the lipid multilayer may also vary depending upon factors such as the degradation rate and the length of time the multilayer is required. For example, the lipid multilayer may vary in diameter from about 10 nm to 50 microns. In some embodiments, the multilayer can have a diameter 50 microns or less, 45 microns or less, 40 microns or less, 35 microns or less, 30 microns or less, 25 microns or less, 20 microns or less, 15 microns or less, 10 microns or less, 5 microns or less, 4 microns or less, 3 microns or less, 2 microns or less, 1 micron or less, 900 nm or less, 800 nm or less, 700 nm or less, 600 nm or less, 500 nm or less, 300 nm or less, 200 nm or less, 100 nm or less, or 50 nm or less. In some embodiments, the multilayer can have a diameter of 10 nm or greater, 20 nm or greater, 30 nm or greater, 40 nm or greater, 50 nm or greater, 75 nm or greater, 100 nm or greater, 200 nm or greater, 500 nm or greater, 750 nm or greater, 1 micron or greater, 2 microns or greater, 3 microns or greater, 4 microns or greater, 5 microns or greater, 7 microns or greater, 8 microns or greater, 10 microns or greater, 15 microns or greater, 20 microns or greater, 30 microns or greater, or 40 microns or greater. In some embodiments, the multilayer can have a diameter of from 10 nm to 50 microns, from 10 nm to 40 microns, from 10 nm to 20 microns, from 10 nm to 10 microns, from 10 nm to 5 microns, from 10 nm to 2 microns, from 10 nm to 1 micron, from 50 nm to 50 microns, from 50 nm to 30 microns, from 50 nm to 10 microns, from 50 nm to 5 microns, or from 50 nm to 1 micron.

The physical characteristics of the lipid multilayer, such as the size, morphology and the amount of silicon containing material present, may be varied or “tuned” depending on the particular make-up of matrix.

The devices disclosed herein can include a plurality of lipid multilayer dots, such as an array of lipid multilayer dots. In some examples, the device can include a second lipid multilayer dot. The second lipid multilayer dodt can comprise a second lipid multilayer structure, wherein the second lipid multilayer structure encapsulates a second material.

The devices disclosed herein can comprise a plurality of cells in contact with the lipid multilayer array.

Methods of Making

Methods of making the devices disclosed herein are provided. In some embodiments, the method can include mixing the lipid with a solvent, such as water and the encapsulated material (such as a hydrophilic or a lipophilic material) to form liposomes. Mixing can be via sonication, centrifugation, or combinations thereof to form the liposomes. In other embodiments, the method can include forming a mixture/solution of the lipid and encapsulated material such as by gently mixing the encapsulated material dissolved in a solvent such as dimethyl sulfoxide (DMSO) with a lipid. The method can further include arraying the liposomes or the mixture/solution of the lipid and encapsulated material onto a support, such as a polydimethylsiloxane (PDMS) pallet. The lipid multilayer arrays can be prepared as disclosed in U.S. Pat. Nos. 9,447,446 and 9,513,222, which are incorporated herein by reference in their entirety. Processes for microarraying lipid multilayers to create spots on a substrate, such as a flat or structured polydimethylsiloxane (PDMS) substrate or “ink-palette” and subsequently transferring these spots into dots by means of multilayer stamping to produce lipid multilayer structures are provided. In one embodiment, a combination of scalable pin-spotting microarray technology with a process of lipid multilayer stamping in order to generate nanostructured lipid multilayer microarrays capable of screening liposomal formulations of encapsulated materials in the dots formed by stamping can be used. In order to improve spot uniformity an ink palette may be used to ink the structured stamp. That is, the inks would be arrayed onto a flat or structured surface, then the structured or flat stamp would be placed in contact with the ink-palette, and finally used for lipid multilayer stamping. The ink-palettes can be dried in a vacuum chamber for 10 minutes to 48 hours to remove unwanted solvents like water or DMSO before the inking and stamping process. Stamping may be used to create spots composed of lipid nanostructures. In the context of lipid multilayer structures formed by stamping, a “spot” is an area of a final patterned surface that originates from a single spot on the ink palette. The finer structures that make up the spot in the resulting array are dots, microstructures or nanostructures. In lipid multilayer stamping, lipids are arrayed onto a structured elastomeric stamp, which is then used to create lipid multilayer patterns. Lipid multilayer stamping techniques that may be used in various embodiments of the present invention are described in U.S. patent application Ser. No. 13/417,588 to Lenhert et al., entitled “Method and apparatus for lipid multilayer patterning,” filed Mar. 12, 2012, and in O. A. Nafday, T. W. Lowry, S. Lenhert, “Multifunctional lipid multilayer stamping,” Small 8(7), 1021-28 (2012), the entire contents and disclosures of which are incorporated herein by reference.

The methods disclosed herein can further include contacting the lipid multilayer dots and/or arrays with a silicon containing material as disclosed herein. The silicon containing material can be in the form of a solution or vapor. The contacting step can be carried out at any suitable temperature such as from 20° C. to 60° C. In some embodiments, the methods include contacting the lipid multilayer dots and/or arrays with a silicon containing material at room temperature and pressure (rtp).

The lipid multilayer dot and/or array can be left in contact with the silicon containing material for greater than 5 minutes up to 24 hours. Preferably, the lipid multilayer dot and/or array can be left in contact with the silicon containing material at room temperature for less than 5 hours, less than 4 hours, less than 3 hours, less than 2 hours, or less than 1 hour.

Pre- and post-immersion uniformity of the lipid multilayers can be determined as described by Lowry et al. (Advanced materials interfaces 1 (4) (2014)). Briefly, an Image J macro was written to define the total lipid pattern area. Next, the pixels were eroded to erase the uniform regions and then dilated to show only non-uniform regions. Percent uniformity of the entire lipid pattern was defined as [1—(non-uniform regions/total area)]×100%.

In specific embodiments, the method can include a) depositing a lipid multilayer array on a surface of a support, wherein the lipid multilayer array comprises one or more lipid multilayer dots, and wherein a hydrophilic material is encapsulated in the one or more lipid multilayer dots, b) contacting a surface of the lipid multilayer dot with a silicon containing precursor, and c) reacting the silicon containing precursor to form a silicon containing coating on the lipid multilayer dot.

In other specific embodiments, the method can include depositing or microarraying lipid droplets on a surface at a temperature of from −10° C. to 30° C. As disclosed herein, the lipid droplet can include an encapsulated material (such as a hydrophilic or hydrophobic therapeutic agent) and a silicon containing coating. The method can further include storing the one or more lipid droplets at a temperature of 10° C. or less for a period of 10 minutes or greater, 30 minutes or greater, 60 minutes or greater, 100 minutes or greater, 5 hours or greater, 10 hours or greater, 12 hours or greater, 24 hours or greater, 36 hours or greater, 48 hours or greater, or from 10 minutes to 48 hours.

The method can include printing the one or more lipid droplets onto a substrate within five of exposure to a temperature greater than 10° C. The lipid droplets can be printed using a topographically structured stamp, as described in U.S. patent application Ser. No. 13/417,588. If the lipid droplets are obtained from storage, preferably the method includes printing the lipid droplets within five minutes of removing the droplets from storage. The topographically structured stamp includes grooves and ridges. In some embodiments, the stamp can be derived from polydimethylsiloxane (PDMS).

In some embodiments, the one or more lipid droplets can be printed by a nanointaglio printing process. For example, the method can include nanointaglio printing the one or more lipid droplets onto a substrate from a topographically structured stamp within five minutes of exposure to a temperature above 10° C.

The method can further include removing the stamp from the substrate to form a patterned substrate.

Methods of Using

Stabilization of surface supported fluid lipid multilayers for underwater characterization is an important step in making them useful for scalable cell culture applications such as high throughput screening. Provided herein are devices that are shown to stabilize fluid lipid films while maintaining their fluidity and functionality under water, to stabilize lipid multilayer micropatterns. The treated multilayers can be immersed under water and successfully imaged by atomic force microscopy (AFM), a difficult feat to perform on untreated fluid lipid multilayers. The silicon based treated lipid multilayer may show an average swelling of about 18% or less in water but can remain stable during the imaging process. The silicon based treated lipid multilayers are also compatible with cell culture such as HeLa, MDCK, and HEK cell types. As such, the devices disclosed herein can be used in biotechnology applications such as microarray based high throughput cell assays.

Also disclosed herein are devices that provide a surface-mediated delivery of an encapsulated material to cells from a microarray of lipid multilayers encapsulating the material. In specific embodiments, the devices disclosed herein are stable in aqueous environments and can be used for delivering of hydrophilic molecules to for example, a cell. In some embodiments, the patterns can successfully deliver an encapsulated material only to the cells directly over them, indicating successful encapsulation and no cross-contamination to cells grown next to the patterns. Accordingly, the efficacies of two drugs can be assayed on the same surface, and it is possible to deliver dosages comparable to those of solution-based delivery (up to the equivalent of 30 μg/mL). Therefore, it is possible to produce a single high-throughput liposome-based screening microarray plate that can be used in the same way as a standard well plate.

The lipid multilayers encapsulating the hydrophilic material, after submerged in water for 100 minutes at 25-37° C., exhibits a leakage of less than 15 wt % of the hydrophilic material originally present in the lipid multilayer array. In some cases, the lipid multilayers encapsulating the hydrophilic material, after submerged in water for 100 minutes at 25-37° C., exhibits a leakage of less than 14 wt %, less than 12 wt %, less than 10 wt %, less than 8 wt %, or less than 5 wt %, of the hydrophilic material originally present in the lipid multilayer array.

To demonstrate the use of lipid multilayer microarrays for delivery to cells, fluorescently labeled lipids and cytotoxic lipophilic or hydrophilic drugs are delivered to the cells and are assayed for toxicity or viral activation. Viral activation can be assayed using doxycycline-induced Karposi Sarcoma endothelial (iSLK) cells.

By way of non-limiting illustration, examples of certain embodiments of the present disclosure are given below.

### EXAMPLES

The following examples are put forth so as to provide those of ordinary skill in the art with a complete disclosure and description of how the compositions and/or methods claimed herein are made and evaluated, and are intended to be purely exemplary and are not intended to limit the scope of the disclosure. Unless indicated otherwise, parts are parts by weight, temperature is in ° C. or is at ambient temperature, and pressure is at or near atmospheric.

### Example 1

### Fluid Lipid Multilayer Stabilization by Tetraethyl Orthosilicate for Underwater AFM Characterization and Cell Culture Applications

EXPERIMENT: 1,2-dioleoyl-sn-glycero-3-phosphocholine (DOPC) and 1,2-dimyristoyl-sn-glycero-3-phosphoethanolamine-N-(lissamine rhodamine-B-sulfonyl) (ammonium salt) or rhodamine-PE dissolved in chloroform were purchased from Avanti Polar Lipids®, aliquoted into a glass vial and dried in a vacuum. Rhodamine-PE was used to dope the DOPC for fluorescence visualization and characterization. Deionized water was added to the vials containing the dried lipids to form liposomes. The samples were sonicated for 10 minutes and aliquoted into microtiter plates. The liposomes were then microarrayed onto a flat polydimethylsiloxane (PDMS) pallet and dried in a vacuum for 2 hours. A PDMS stamp with micro-well features of 5 μm diameter and 2.5 μm depth, covering 19% of the stamp surface, was inked by pressing the patterned surface onto the microarrayed pallet (T. W. Lowry et al., Advanced materials interfaces 1 (4) (2014)). The inked PDMS stamp was then pressed onto clean glass slides to obtain discrete 5 μm diameter dots.

Two glass vials, one containing 2 mL of TEOS, purchased from Sigma Aldrich® and the other containing 0.1 M HCl were placed into a larger glass container. A glass slide with the printed lipid multilayers was also placed in the larger glass container. The container was then sealed tight and placed in an oven at 60° C. for 2 hours after which the samples were ready for use.

Specifically, the method for encapsulation of hydrophilic molecules for microarray and nanointaglio printing include aliquoting lipids into glass vial followed by drying. Adding an aqueous solution of drug to the dried vial, sonicating, centrifuging for 10 minutes at 14 rpm and aliquoting the supernatant into a microtiter plate for microarraying. For hydrophobic drugs, the method includes aliquoting a mixture of drugs and lipids into a glass vial followed by drying. Adding an appropriate biological buffer to the dried vial, sonicating, and aliquoting a solution into a microtiter plate for microarraying.

The method for TMOS/TEOS treatment of lipid/drug multilayers includes drying and stamping microarrayed pallets onto glass substrate using the nanointaglio printing method. Placing sample in a large glass container with TMOS and sealing the container. Incubating the sample for between 30 minutes to 4 hours. Placing the sample in a vacuum for between 15 minutes to 24 hours to remove residual condensed TEOS/TMOS. Placing the sample in ambient humidity (40-80% RH) 20 minutes to 2 hours. Pipetting solution of cells onto array sample

Immersion of lipid multilayers under low humidity was done in a nitrogen glove box with humidity set to <2 ppm. Any other immersion was done under ambient conditions. All immersion for AFM was done by gently pipetting a drop of the buffer directly over each pattern. For the overnight humidity study, the TEOS-treated SMLs were left on the benchtop at ambient conditions for 12 hours. Culture media without phenol red was used for media immersions.

Pre- and post-immersion uniformity of the lipid multilayers was measured as described by Lowry et al.. Briefly, an Image J macro was written to define the total lipid pattern area. Next, the pixels were eroded to erase the uniform regions and then dilated to show only non-uniform regions. Percent uniformity of the entire lipid pattern was defined as [1—(non-uniform regions/total area)]×100%.

AFM of the lipid prints was done using peak force mode with a fluid cell cantilever holder and protective skirt at 512 samples per line with a Dimension Icon AFM (Bruker, Billerica, Mass., USA) and peak force mode cantilevers (Scanasyst Fluid+, 2 nm nominal tip radius, 2.5-8.0 μm tip height, 0.7 N/m spring constant, Bruker, Millerica, Mass., USA), respectively. Scan frequencies of 0.300 Hz or 0.500 Hz were used for high resolution air and underwater AFM imaging.

The cells used for the experiments (Hela, Madin-Darby Canine Kidney Epithelial (MDCK) and HEK 293-AAV) were purchased from American Type Culture Collection (ATCC), Manassas, Va., USA. The cells were grown to 70% confluency 24 hours before use. The cells were gently trypsinized and seeded over the multilayer microarrays by adding 2 mL of the cell suspension at a density of 200,000 cells/mL. The cells were incubated over 12 hours for the adhesion study. The cells were then washed with Hank's Balanced Salt (HBSS) buffer, DAPI stained by incubating the cells with the dye for 10 minutes and then washed. The lipid multilayers and the stained cells were imaged using a Nikon Eclipse Ti fluorescent microscope equipped with the Nikon G-2E/C and UV-2E/C fluorescent filters.

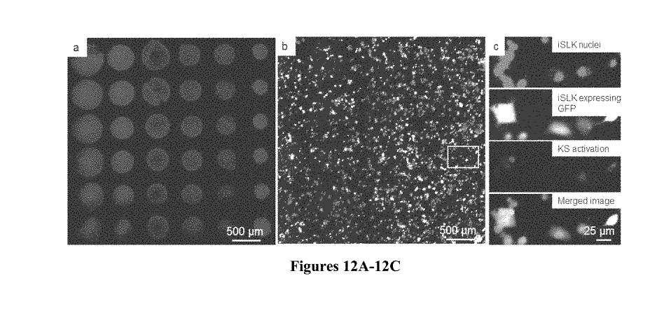

To demonstrate the use of lipid multilayer microarrays for delivery to cells, fluorescently labeled lipids and cytotoxic lipophilic or hydrophilic drugs were delivered to the cells and assayed for viral activation. Viral activation was assayed using doxycycline-induced Karposi Sarcoma endothelial (iSLK) cells indicated by the fluorescence protein (GFP) expressed in the cytoplasm and the stained nuclei in FIGS. 12B and 12C. Viral expression is indicated by the fluorescent protein (RFP) used as the reporter gene. This is also shown in FIGS. 12B-12C.

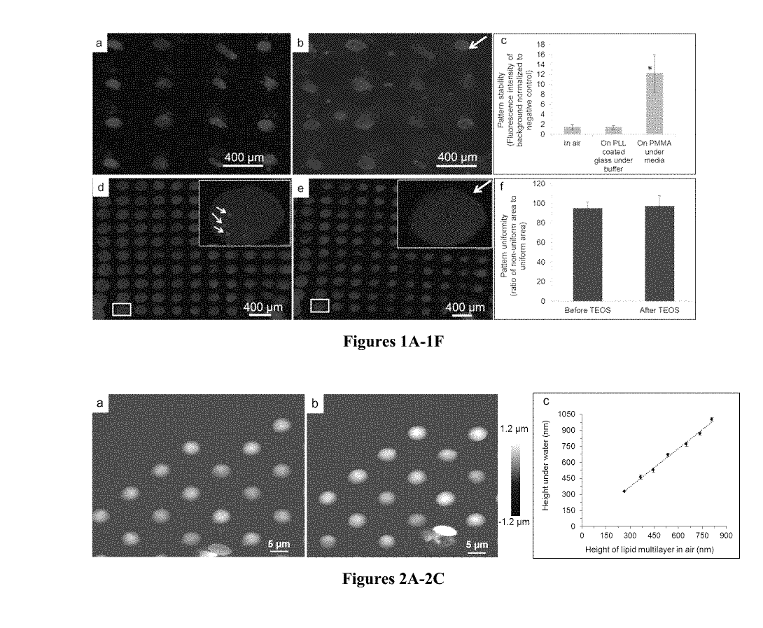

RESULTS AND DISCUSSION: Fluid rhodamine-PE doped DOPC multilayer arrays shown in FIG. 1A using a combination of the microarray and nanointaglio printing methods were fabricated (T. W. Lowry et al.). FIG. 1B shows the destruction of the pattern in FIG. 1A upon immersion in cell culture media under low humidity conditions (<2 ppm). The destruction was quantified by measuring the fluorescence intensity of the intervening areas between the arrayed multilayers as it indicated the dissolution and deposition of the dissolved lipids onto previously empty areas (FIG. 1C). Treatment of lipid multilayers in FIG. 1D with TEOS stabilized them for aqueous immersion as shown in FIG. 1E. The pattern stability of untreated multilayers to the immersed, TEOS treated multilayers was compared by measuring the pre and post-immersion pattern uniformity. Uniformity, defined here as the percentage of a spot that is made up of discrete 5 μm dots rather than larger contiguous multilayers was determined using the method developed by Lowry et al. The pattern fidelity post TEOS treatment and immersion to be as high as pre-TEOS treatment was determined. This is essential for biological applications where cross-contamination is undesirable.

Next, the heights of the same lipid multilayers before and after TEOS treatment using AFM were measured. FIG. 2A shows the AFM peak force mode images of TEOS lipid multilayers in air while FIG. 2B shows the same area under water. The graph in FIG. 2C shows an approximately 18% swelling of the multilayers about 30 minutes after immersion. The stability of the dots indicates a potential use for encapsulating materials without loss of the encapsulated materials into solution during immersion.

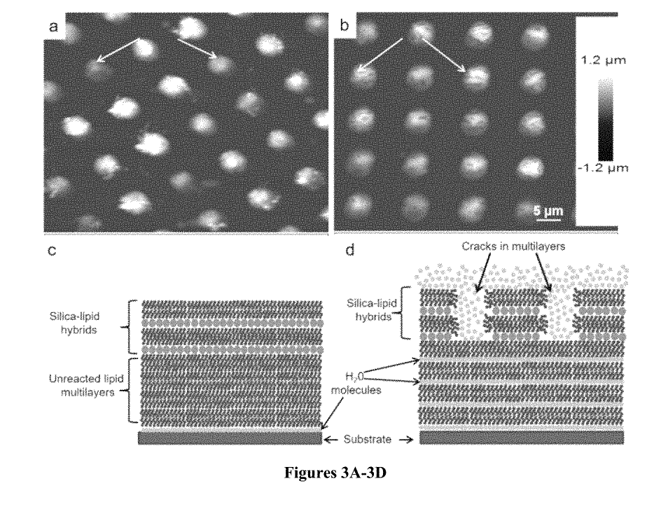

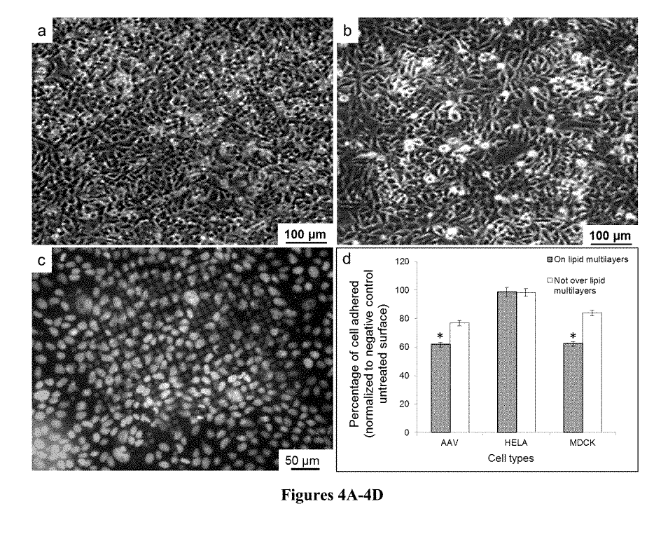

In order to determine the suitability of the TEOS-treated lipid multilayers to biological and biochemical applications, the effect of extended exposure to humidity and liquid water was studied. When exposed to humidity overnight, the treated multilayers showed cracks on the surfaces as shown with the arrows in FIG. 3A as compared to FIG. 2B. The cracks observed in the multilayers are consistent with literature where TEOS treatment is done at 60° C. (G. Gupta et al. ACS Nano 7, 5300-5307 (2013)). These cracks did not appear when the reaction was performed at room temperature (G. Gupta et al.). It was postulate that the cracks appear in the harder silica-phospholipid hybrid outer shell of the multilayer as a result of relatively greater expansion of the lipid multilayers upon absorption of water molecules after TEOS treatment at 60° C. compared to treatment at room temperature (G. Gupta et al. and U. Y. Wang et al., J. Phys. Chem. B 108, 4767-4774 (2004)). The same cracks shown in the schematic of FIG. 3A appear in the multilayers immersed under buffer for 2 hours as shown in FIG. 3B. While these cracks indicate that the outer shell can lose some of its integrity, the cracks appear well after immersion and can be avoided entirely by treatment at a lower temperature (G. Gupta et al.). The lipid multilayers themselves are stable enough in biological buffer and media to prevent cross-contamination of adjacent spots (S. N. Bailey et al, Proc. Natl. Acad. Sci. U.S.A. 101, 16144-16149 (2004)).

Finally, the suitability of the TEOS-treated lipid multilayers for cell culture was tested by measuring adhesion of various cell types onto the patterns. Cell adhesion was chosen because it is the initial step necessary for any surface-based live cell assay. Hela, MDCK and the AAV variant of the HEK 293 cell lines were used. FIGS. 4A and 4B show high adhesion over both TEOS treated and untreated lipid multilayers. FIG. 4C shows a sample spot of TEOS-treated lipid multilayers made up of dots with sub-cellular lateral dimensions with cells growing over them. In FIG. 4D the efficiency of cellular attachment as the percentage of cells adhered to the treated surface when compared to an untreated surface without lipids was quantified. The Hela cells showed the highest level of adhesion to both the TEOS-treated lipid multilayers and the spaces without lipids. The ubiquitous adhesion of Hela cells on and off the lipid patterns is especially useful as the intervening areas without lipids can be used as control areas within test samples. Both the MDCK and AAV cells showed a significant difference in adhesion between the patterned and unpatterned areas within the same sample in addition to being lower than the negative control with no treatment. These differences can be easily accounted for during experimentation using multilayers without any test molecules as a negative control. The result also indicates that each cell type might respond differently and therefore appropriate controls should be applied when using them for surface-based assays.

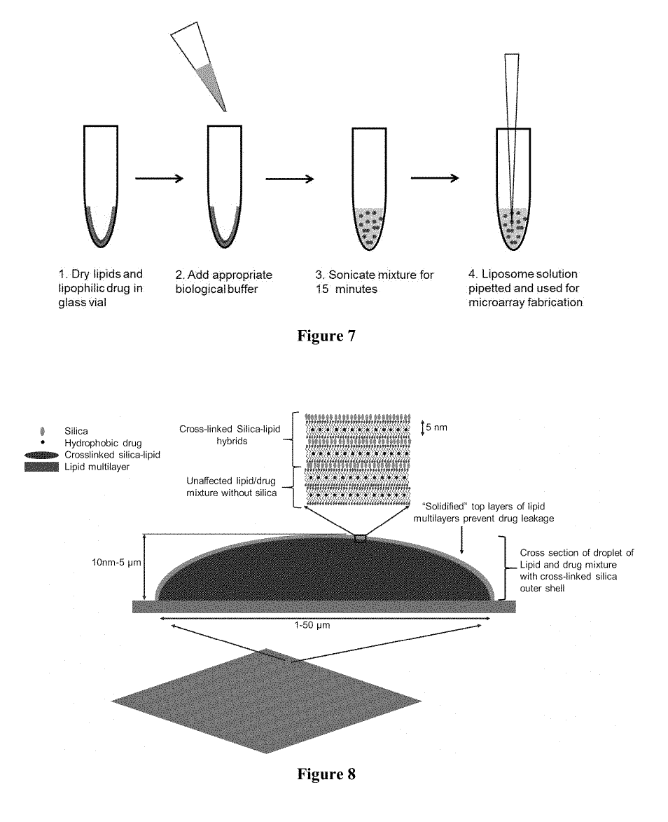

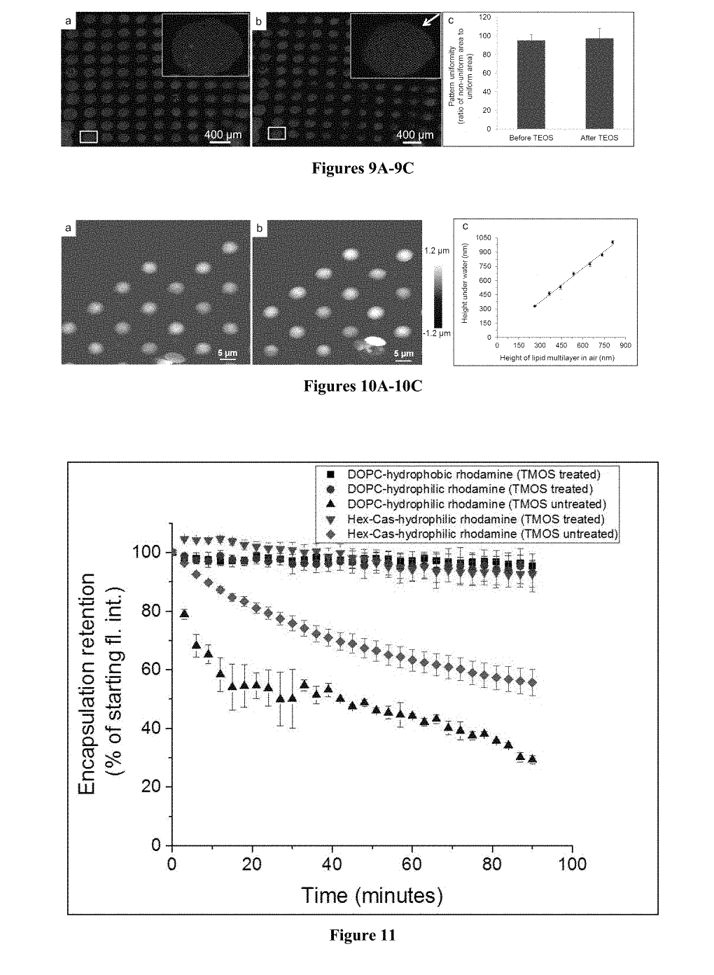

FIGS. 5-8 shows schematic of encapsulation of hydrophilic and hydrophobic molecules for microarray printing as well as stabilization of lipid multilayers with the encapsulated molecules.

SUMMARY: Treatment of surface supported fluid lipid multilayers with TEOS stabilized the lipids to allow for underwater AFM imaging. The treated lipid multilayers also lend themselves to cell culture applications (e.g., cellular uptake, high throughput screening, and characterization of lipid membranes and other lipid based bio-systems) as cells adhere to the lipid patterned areas.

Embodiments of the Invention

A device comprising, a support, a discrete lipid multilayer array on a surface of the support, wherein the lipid multilayer array comprises one or more surface supported lipid multilayer dots, a hydrophilic or hydrophobic material encapsulated in the one or more lipid multilayer dots, and a silicon containing compound present on a surface of each lipid multilayer dot.

The device of the preceding embodiment, wherein the encapsulated material is hydrophilic.

The device of any one of the preceding embodiments, wherein the encapsulated material is hydrophilic.

The device of any one of the preceding embodiments, wherein the silicon containing compound is silica based.

The device of any one of the preceding embodiments, wherein the silica based compound is derived from an alkyl silicate.

The device of any one of the preceding embodiments, wherein the alkyl silicate is selected from tetramethyl orthosilicate, tetraethyl orthosilicate, tetraisopropyl orthosilicate, tetrapropyl orthosilicate, or combinations thereof.

The device of any one of the preceding embodiments, wherein the silicon containing compound forms a lipid-silicon based hybrid assembly on the surface of the lipid multilayer dot.

The device of any one of the preceding embodiments, wherein the silicon containing compound is present in an amount of from greater than 0 wt % to 70 wt %, based on the weight of each lipid multilayer dot.

The device of any one of the preceding embodiments, wherein the hydrophilic material is a drug.

The device of any one of the preceding embodiments, wherein the drug is a small molecule.

The device of any one of the preceding embodiments, wherein the device comprises a plurality of the lipid multilayer dots, wherein the lipid multilayer dots are discrete and separated by spaces on the support.

The device of any one of the preceding embodiments, wherein each of the lipid multilayer dot has a height of 50 μm or less.

The device of any one of the preceding embodiments, wherein the height of the lipid multilayer dot is from 10 nm to 50 μm.

The device of any one of the preceding embodiments, comprising a plurality of the discrete lipid multilayer arrays separated from each other by spaces on the support.

The device of any one of the preceding embodiments, wherein the plurality of the discrete lipid multilayer arrays comprise a second lipid multilayer array, wherein the second lipid multilayer array comprises one or more surface supported second lipid multilayer dots, and wherein each of the second lipid multilayer dot encapsulates a second material.

The device of any one of the preceding embodiments, wherein the device further comprises a labeling material or a targeting agent.

The device of any one of the preceding embodiments, wherein the device comprises a plurality of cells in contact with the lipid multilayer array.

The device of any one of the preceding embodiments, wherein the device after submerged in water for 100 minutes at from 25° C. to 37° C., exhibits a leakage of less than 15 wt % of the hydrophilic material originally encapsulated in the lipid multilayer dot.

A method of producing a device comprising, depositing a lipid multilayer array on a surface of a support, wherein the lipid multilayer array comprises one or more lipid multilayer dots, and wherein a hydrophilic material is encapsulated in the one or more lipid multilayer dots, contacting a surface of the lipid multilayer dot with a silicon containing precursor, and reacting the silicon containing precursor to form a silicon containing coating on the lipid multilayer dot.

The method of any one of the preceding embodiments, wherein the silicon containing precursor is a silicate based compound.

The method of any one of the preceding embodiments, wherein the silicate based compound is an alkyl silicate.

The method of any one of the preceding embodiments, wherein the alkyl silicate is selected from tetramethyl orthosilicate, tetraethyl orthosilicate, tetraisopropyl orthosilicate, tetrapropyl orthosilicate, or combinations thereof.

The method of any one of the preceding embodiments, wherein the silicon containing precursor is in the form of a solution or vapor.

The method of any one of the preceding embodiments, wherein step b) contacting the surface of the lipid multilayer dot with the silicon containing precursor is performed at room temperature.

A method for delivering a hydrophilic material comprising, providing a device of any one of embodiments 1-16, comprising a lipid multilayer dot, delivering the hydrophilic material to a cell from the lipid multilayer dot that is in contact with the cell.

A device comprising, a support, a discrete lipid multilayer array on a surface of the support, wherein the lipid multilayer array comprises one or more lipid multilayer dots, a therapeutic agent encapsulated in the one or more lipid multilayer dots, a silica based compound present on a surface of the one or more lipid multilayer dots.

The device of any one of the preceding embodiments, wherein the silica based compound is derived from an alkyl silicate selected from tetramethyl orthosilicate, tetraethyl orthosilicate, tetraisopropyl orthosilicate, tetrapropyl orthosilicate, or combinations thereof.

The device of any one of the preceding embodiments, wherein the silicon containing compound forms a lipid-silicon based hybrid assembly on the surface of the lipid multilayer dot.

The device of any one of the preceding embodiments, wherein the therapeutic agent is a hydrophilic small molecule.

The device of any one of the preceding embodiments, wherein the device further comprises a second lipid multilayer array, wherein the second lipid multilayer array comprises one or more second lipid multilayer dots, and wherein the one or more second lipid multilayer dots encapsulate a second material.

The device of any one of the preceding embodiments, wherein the device after submerged in water for 100 minutes at from 25° C. to 37° C., exhibits a leakage of less than 15 wt % of the material originally encapsulated in the lipid multilayer dot.

A method of producing a device comprising, depositing one or more lipid droplets on a surface at a temperature of from −10° C. to 30° C., wherein the lipid droplet comprises a therapeutic material and a silicon containing coating; storing the one or more lipid droplets at a temperature of 10° C. or less for a period of at least 10 minutes such as from 10 minutes to 48 hours; printing using a nanointaglio process the one or more lipid droplets on a substrate using a topographically structured stamp within five minutes of exposure to a temperature above 10° C.; and removing the stamp from the substrate to form a patterned substrate.

The method of any one of the preceding embodiments, wherein the stamp is derived from polydimethylsiloxane (PDMS).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative materials and method steps disclosed herein are specifically described, other combinations of the materials and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A device comprising, a support,
a discrete lipid multilayer array on a surface of the support, wherein the lipid multilayer array comprises one or more lipid multilayer dots,
a material encapsulated in the one or more lipid multilayer dots, and
a silicon containing compound present on a surface of each lipid multilayer dot.

2. The device of claim 1, wherein the silicon containing compound is derived from an alkyl silicate.

3. The device of claim 1, wherein the silicon containing compound forms a lipid-silicon based hybrid assembly on the surface of the lipid multilayer dot.

4. The device of claim 1, wherein the silicon containing compound is present in an amount of from greater than 0 wt % to 70 wt %, based on the weight of each lipid multilayer dot.

5. The device of claim 1, wherein the material encapsulated in the one or more lipid multilayer dots is a therapeutic agent.

6. The device of claim 1, wherein each lipid multilayer dot has a height of from 10 nm to 50 μm.

7. The device of claim 1, comprising a plurality of the discrete lipid multilayer arrays separated from each other by spaces on the support.

8. The device of claim 1, comprising a plurality of discrete lipid multilayer arrays, wherein the discrete lipid multilayer arrays comprise a second lipid multilayer array,
wherein the second lipid multilayer array comprises one or more surface supported second lipid multilayer dots, and wherein each of the second lipid multilayer dot encapsulates a second material.

9. The device of claim 1, wherein the device comprises a plurality of cells in contact with the lipid multilayer array.

10. The device of claim 1, wherein the device after submerged in water for 100 minutes at from 25° C. to 37° C., exhibits a leakage of less than 15 wt % of a hydrophilic material originally encapsulated in the lipid multilayer dot.

11. A method of producing a device comprising,
a) depositing a lipid multilayer array on a surface of a support, wherein the lipid multilayer array comprises one or more lipid multilayer dots, and wherein a hydrophilic material is encapsulated in the one or more lipid multilayer dots,
b) contacting a surface of the lipid multilayer dot with a silicon containing precursor, and
c) reacting the silicon containing precursor to form a silicon containing coating on the lipid multilayer dot.

12. The method of claim 11, wherein the silicon containing precursor is an alkyl silicate.

13. The method of claim 11, wherein the silicon containing precursor is in the form of a solution or vapor.

14. The method of claim 11, wherein step b) contacting the surface of the lipid multilayer dot with the silicon containing precursor is performed at room temperature.

15. A method for delivering a hydrophilic material comprising,
providing a device of claim 1 comprising a lipid multilayer dot,
delivering the encapsulated material to a cell from the lipid multilayer dot that is in contact with the cell.

16. The device of claim 1, comprising:
the support,
the discrete lipid multilayer array on a surface of the support, wherein the lipid multilayer array comprises one or more surface supported lipid multilayer dots,
a therapeutic agent encapsulated in the one or more lipid multilayer dots,
the silica based compound present on a surface of the one or more lipid multilayer dots.

17. The device of claim 16, wherein the therapeutic agent is a hydrophilic small molecule.

18. The device of claim 16, wherein the device further comprises a second lipid multilayer array,
wherein the second lipid multilayer array comprises one or more second lipid multilayer dots, and wherein the one or more second lipid multilayer dots encapsulate a second material.

19. A method of producing a device comprising,
a) depositing one or more lipid droplets on a surface at a temperature of from −10° C. to 30° C., wherein the lipid droplet comprises a therapeutic material and a silicon containing coating;
b) storing the one or more lipid droplets at a temperature of 10° C. or less for a period of at least 10 minutes;
c) printing using a nanointaglio process the one or more lipid droplets from step (b) on a substrate using a topographically structured stamp within five minutes of exposure to a temperature above 10° C.; and
d) removing the stamp from the substrate to form a patterned substrate.

20. The method of claim 19, wherein the stamp is derived from polydimethylsiloxane (PDMS).
